(19) United States
(12) Patent Application Publication
(10) Pub. No.: US 2018/0125842 A1
(43) Pub. Date: May 10, 2018

(54) Ergoloid for Cancer

(71) Applicants: Ronald Blair Hartman, Leawood, KS (US); Renee Marie Hartman, Leawood, KS (US)

(72) Inventors: Ronald Blair Hartman, Leawood, KS (US); Renee Marie Hartman, Leawood, KS (US)

(21) Appl. No.: 15/348,209

(22) Filed: Nov. 10, 2016

(51) Int. Cl. A61K 31/4985; A61K 45/06; A61K 31/09; A61K 31/353

(52) U.S. Cl. CPC A61K 31/4985; A61K 31/353; A61K 31/09; A61K 45/06

(57) ABSTRACT

A drug developed to treat dementia which has been abandoned by its manufacturer, Novartis, is repurposed to enhance the treatment of cancer. The drug, Ergoloid, contains four gamma secretase inhibitors that produce the same benefit as single gamma secretase inhibitors that are in clinical trials to enhance the treatment of cancer. The method of this invention is to administer Ergoluid 1 mg three times a day by mouth.

This enhancement results in better outcomes with lower cost and better quality of life.

### CROSS-REFERENCE TO RELATED APPLICATIONS

none

### STATEMENT REGARDING FEDERALLY SPONSORED RESEARCH

none

### BACKGROUND OF INVENTION

Ergoloid was marketed for the treatment of dementia, hypertension, stroke and prolactinoma by Novartis. The FDA approved this drug in 1982. The patent on this medication issued in 1988—has expired. (U.S. Pat. No. 4,737,499). U.S. Pat. No. 4,737,499 contains a detailed and precise specification of this compound

Ergoloid was studied in over 150 clinical trials. There is no prior art suggesting usefulness in treating cancer, and no published literature suggesting any usefulness in treating cancer. Thus our invention of using Ergoloid to enhance cancer treatment is non-obvious and completely new. It is based on actual human clinical use.

Ergoloid has no effect on cancer by itself Which may account for the failure to notice its usefulness in this application. It must be paired with a cancer treatment to notice its effects.

Our invention is the application of Ergoloid in conjunction with active cancer treatments. We have used Ergoloid in colorectal cancer in conjunction with preoperative radiation and chemotherapy inducing complete remission of the bulky tumor and lymph node involvement avoiding the need for major colon surgery and following chemotherapy. We have used Ergoloid in patients where pterostilbene has failed, after years of use, and the subsequent Lupron/Casodex treatment for prostate cancer caused stroke, to put the patient back on the low toxicity pterostilbene (to which the tumor had previously become resistant) with complete remission of the cancer. We have used Ergoloid in conjunction with Rituxan, an immunotherapy, in splenic lymphoma where Rituxan was gradually allowing progression, to restore the treatment to its initial success. We have used Ergoloid in non-small cell lung cancer. This allowed a stage IV B patient to regain his lost weight, and continue working in spite of undergoing treatment for lung cancer.

The least toxic combination we have used so far is Ergoloid/pterostilbene.

(Patent WO2011042482A1 covers the use of pterostilbene in solid tumors alone or in conjunction with chemotherapy, and/or radiotherapy. It makes no mention of using Ergoloid with pterostilbene.)

Pterostilbene fails in the same fashion as platinum chemotherapy, (“Cisplatin selects for Multi-Drug Resistant Cell in Lung Adenocarcinoma by Activating Notch 1 Signaling” Cancer Research 73(1) 1-11 2012 Liu et al. “Pterostilbene Exerts Antitumor action via Notch 1 signaling pathway in Human Lung Adenocarcinoma Cells” PLOS One 8(5):e62652 Yang et al.) This the same pathway by which hormone therapy, immunotherapy, and radiotherapy fail by inducing Notch 1 signaling pathway.

Ergoloid can restore failed therapies, and can enhance initial treatments. Yet Ergoloid's gamma secretase inhibiting components individually fail the standard assay for Notch 1 inhibition. They do succeed in the APP cleavage assay for gamma secretase inhibition activity in Alzheimer's disease. (“The FDA approved natural product dihydroergocristine reduce the Production of the Alzheimer's Disease Amyloid beta Peptides” Science Reports 2015 nov PMCID:PMC4644980 Lei et al.)

Thus, most people would not investigate further vis a vis cancer treatment.

In a flash of inspiration, I realized that four gamma secretase inhibitors contained in Ergoloid, that failed the standard Notch 1 assay individually, might still combine to produce enough Notch 1 inhibition when used together. As noted above, this flash of inspiration proved to be correct with Ergoloid delivering the benefits of Notch 1 inhibition without the toxicity. (“The cautionary tale of chronic Notch 1 inhibition” Journal of Clinical Investigation Vol. 121 no. 2 pp 508-509 Sandra Rayeom)

Unlike the other current gamma secretase inhibitors in clinical trials which have major toxicity comparable to chemotherapy agents, Ergoloid has only minor nausea and headache, rarely, with no impact on bone marrow; it does not induce tumors in the GI tract unlike other gamma secretase inhibitors currently in clinical trials, and it does not cause vomiting, weight loss, or extreme weakness and fatigue. This represents a significant improvement in quality of life for a patient requiring cancer treatment. Ergoloid used to enhance treatment is a major breakthrough in cost reduction as well. For instance, Lupron/Casodex treatment regimen for prostate cancer cost approximately $40,000 per year and will usually fail after two years of treatment. Ergoloid/pterostilbene costs less than $4000 per year and will usually induce complete remission in 4 months with no risk of stroke, heart attack, or congestive heart failure. In other words, use of Ergoloid holds the potential to reduce treatment costs by 90% or more and improve the patients' quality of life. For this reason we foresee the day when payers will require the use of treatments like this before approving more expensive, less effective, and more toxic therapies.

1. (canceled)

2. (canceled)

3. (canceled)

4. (canceled)

5. A method of treatment of cancer by administering Ergoloid in combination with anti-cancer treatment.

6. The method of claim 5 wherein the cancer is one of the following cancers: pancreatic cancer, lung cancer, breast cancer, colon cancer, rectal cancer, prostate cancer, lymphoma.

7. The method of claim 5 wherein the cancer treatment is provided by one or more of the following anti cancer agents: radiation, chemotherapy, immunotherapy, hormone therapy, quercetin, pterostilbenc.

8. The method of claim 1 where the original anti-cancer treatment has failed and Ergoloid is added to restore effectiveness of the failed anticancer treatment.
